(19) United States
(12) Patent Application Publication
(10) Pub. No.: US 2026/0345528 A1
(43) Pub. Date: Nov. 5, 2026

(54) WATER-SOLUBLE CARRIER-BASED HIGH-CONCENTRATION DOXYCYCLINE COMPOSITION FOR PERIODONTAL DISEASE TREATMENT

(71) Applicant: Dong Yeol JUNG, Gyeonggi-do (KR)

(72) Inventor: Dong Yeol JUNG, Gyeonggi-do (KR)

(21) Appl. No.: 19/286,469

(22) Filed: Jul. 31, 2025

(30) Foreign Application Priority Data
Apr. 22, 2025 (KR) 10-2025-0052559

(51) Int. Cl. A61K 31/165; A61K 9/00; A61K 47/36; A61K 47/44; A61P 1/02

(52) U.S. Cl. CPC A61K 31/165; A61K 9/0053; A61K 47/36; A61K 47/44; A61P 1/02

(57) ABSTRACT

The purpose is to provide a pharmaceutical composition for treating periodontal disease containing high-concentration doxycycline, where a carrier that delivers antibiotics to the treatment site in the oral cavity can be decomposed in a short period. For this purpose, a water-soluble carrier-based high-concentration doxycycline-containing pharmaceutical composition for periodontal disease treatment is provided, comprising 20 to 30 weight % of doxycycline and 70 to 80 weight % of an oral water-soluble carrier. According to this, since the oral water-soluble carrier that delivers doxycycline to the treatment site is completely decomposed within 72 hours in the oral cavity, the problem of the carrier interfering with periodontitis healing by remaining around the gums for a long time is solved.

### BACKGROUND

The present invention relates to a pharmaceutical composition for treating periodontal disease containing high-concentration doxycycline to improve periodontal diseases occurring in the oral cavity. More particularly, the present invention relates to a water-soluble carrier-based high-concentration doxycycline-containing pharmaceutical composition for periodontal disease treatment, which not only effectively delivers antibiotic components to the treatment site using a water-soluble carrier but also prevents inhibition of granulation tissue formation by being rapidly removed, and effectively treats periodontal diseases such as periodontitis using high-concentration doxycycline.

Periodontal disease, also known as pyorrhea, includes gingivitis, which is an early stage disease where inflammation occurs in the gums, and periodontitis, which is a stage where inflammation progresses not only to the gums but also to the bone and tissues supporting the teeth.

One of the main causes of such periodontitis is known to be the action of bacteria (microorganisms) that constitute biofilms formed on the surface of teeth and gums. That is, oral bacteria attach to the surface of teeth and form a membrane called a biofilm. Toxins released from this biofilm cause inflammatory reactions in tissues around the gums, which is the main cause of periodontal diseases including periodontitis, and it can also occur in the form of peri-implantitis when post-implant care is neglected.

Preventively, regular dental care such as brushing teeth or scaling to prevent biofilm formation is important. However, if it has already progressed to acute or chronic periodontitis, treatment to remove bacteria from tissues around the teeth, including the periodontal pocket, with antibiotics is necessary.

Various antibiotics have been used for the treatment of periodontitis. In particular, tetracycline-class antibiotics such as minocycline and doxycycline have excellent cation affinity, making them effective for peri-implantitis, and have excellent lipid solubility, allowing them to easily penetrate bacterial membranes (phospholipids/lipid-soluble). They also have acidic properties, making them excellent at removing smear layers that interfere with healing, such as bacteria, saliva, blood, and impurity films on the tooth surface.

In particular, Korean Patent No. 10-2657091 (Title of Invention: Pharmaceutical Composition for Periodontitis Treatment Containing High-Concentration Tetracycline-Class Antibiotics for Removal of Harmful Oral Bacteria and Restoration of Oral Microbial Ecosystem) registered by the present inventor and a paper (Jung D Y, Cha J K, Kim Y T, Kim C S. Comparing the clinical effects of local administration of minocycline ointment and doxycycline solution in the treatment of acute periodontal abscesses: a retrospective clinical study. J Periodontal Implant Sci. 2023; 53(5): 289-304. Published online Sep. 11, 2023. doi:10.5051/jpis.2305300180) disclose treating periodontitis using high-concentration doxycycline.

Specifically, according to the registered patent and the paper (Jung et al.), local application of doxycycline solution with drainage is more effective than minocycline ointment in treating acute periodontal abscesses, and local application of doxycycline solution (300 mg/mL, which corresponds to approximately 30% concentration) is more effective than minocycline ointment (2%) in treating acute periodontal abscesses.

These studies are merely confirmations of short-term therapeutic effects of about 4 weeks during the treatment process that proceeds from hemostasis phase-inflammation phase-proliferation phase-maturation phase, so additional observation of the degree of regeneration of periodontal ligaments after a long period (long-term), up to more than 1 year, is needed.

Furthermore, additional research is needed on the effective concentration range of doxycycline for long-term regeneration of teeth and surrounding tissues after periodontitis treatment, as well as the role of the base (carrier) that delivers antibiotics, its side effects, and how to control these side effects.

### PATENT DOCUMENTS

(Patent Document 1) Korean Patent No. 10-2657091 (Published on Apr. 15, 2024)
(Patent Document 2) Korean Patent No. 10-1406106 (Published on Jun. 13, 2014)
(Patent Document 3) Korean Patent Application Publication No. 10-2003-0006788 (Published on Jan. 23, 2003)

### Non-Patent Document

(Paper 1) Jung D Y, Cha J K, Kim Y T, Kim C S. Comparing the clinical effects of local administration of minocycline ointment and doxycycline solution in the treatment of acute periodontal abscesses: a retrospective clinical study. J Periodontal Implant Sci. 2023; 53(5):289-304. Published online Sep. 11, 2023. doi: 10.5051/jpis.2305300180

### SUMMARY OF THE INVENTION

Therefore, the purpose of the present invention is to identify the effective concentration range of doxycycline that can effectively treat periodontal diseases such as periodontitis, and to provide a pharmaceutical composition for treating periodontal disease containing high-concentration doxycycline with a water-soluble carrier base that can solve the problem of carriers interfering with healing by remaining in the gingiva for more than 2 weeks when delivering antibiotics to the treatment site in the oral cavity.

To achieve the above-mentioned purpose of the present invention, one embodiment of the present invention provides a water-soluble carrier-based high-concentration doxycycline-containing pharmaceutical composition for periodontal disease treatment, comprising 20 to 30 weight % of doxycycline and 70 to 80 weight % of an oral water-soluble carrier.

To achieve the above-mentioned purpose of the present invention, a more preferred embodiment of the present invention provides a water-soluble carrier-based high-concentration doxycycline-containing pharmaceutical composition for periodontal disease treatment, comprising 25 to 30 weight % of doxycycline and 70 to 75 weight % of an oral water-soluble carrier.

According to the present invention, the oral water-soluble carrier that performs the role of delivering doxycycline to the treatment site can disappear by decomposing in the oral cavity in about 10 minutes and disappears by decomposing within 72 hours at the latest. Therefore, the problem of the carrier interfering with the action of macrophages or the formation and proliferation of granulation tissue by remaining around the gums for a long time is solved.

In particular, since the carrier used in the present invention is water-soluble, it can be easily washed away with water, etc., without complex treatment or use of drugs, and through this, it is almost completely removed.

In addition, since the pharmaceutical composition for treating periodontal disease according to the present invention is effective in removing both Gram-positive and Gram-negative bacteria unlike minocycline, it can be used for the treatment of gingivitis as well as for the treatment of periodontitis.

Furthermore, the pharmaceutical composition for treating periodontal disease according to the present invention can be manufactured as a liquid or ointment form of solid preparation, improving convenience of treatment, and can provide quick and effective treatment opportunities for patients with periodontitis, peri-implantitis, periodontal abscess, periapical lesions (endodontic treatment), etc. Therefore, it can ultimately improve the quality of life of patients and provide help in terms of time and economy.

### BRIEF DESCRIPTION OF THE DRAWINGS

FIGS. 1 to 7 are comparative photographs showing changes before and after use of the pharmaceutical composition for treating periodontal disease according to one embodiment of the present invention.

FIGS. 8 to 14 are comparative photographs showing changes before and after use of the pharmaceutical composition for treating periodontal disease according to a comparative embodiment of the present invention.

FIG. 15 is a graph showing the average recovery rate by concentration of doxycycline included in the pharmaceutical composition for treating periodontal disease according to the present invention.

### DETAILED DESCRIPTION OF THE INVENTION

Hereinafter, a water-soluble carrier-based high-concentration doxycycline-containing pharmaceutical composition for periodontal disease treatment (hereinafter abbreviated as ‘periodontal disease treatment composition’) according to preferred embodiments of the present invention will be described in detail with reference to the accompanying drawings.

The periodontal disease treatment composition according to the present invention is used on the affected area after incising or non-incising the affected gum and removing inflammation existing in the periodontal pocket, and includes 20 to 30 weight % of doxycycline and 70 to 80 weight % of an oral water-soluble carrier, and may optionally further include additives. It is preferable that such a periodontal disease treatment composition includes 25 to 30 weight % of doxycycline and 70 to 75 weight % of an oral water-soluble carrier.

Hereinafter, the periodontal disease treatment composition of the present invention will be described more specifically for each component.

The periodontal disease treatment composition according to the present invention includes doxycycline. The doxycycline is used as an antibiotic for the treatment of periodontal disease and provides additional effects such as anti-inflammatory action and prevention of alveolar bone resorption in addition to antibacterial effects.

The doxycycline is an antibiotic of the tetracycline class along with tetracycline and minocycline, with a molecular formula of C22H24N2O8 and a molecular weight of 444.440 g/mol.

The doxycycline has a half-life of 15-25 hours and is more lipid-soluble than tetracycline, so it not only penetrates mucosal tissues better but also has a relatively longer-lasting effect, making it used for the treatment of periodontal diseases such as gingivitis and periodontitis.

The doxycycline has the advantage of reduced risk of tooth discoloration and fewer side effects compared to minocycline in the treatment of acute periodontitis.

As such, doxycycline has superior advantages as an oral antibiotic compared to tetracycline or minocycline.

Such doxycycline can be added at 20 to 30 weight %, preferably 25 to 30 weight %, based on 100 weight % of the periodontal disease treatment composition. The periodontal disease treatment composition containing doxycycline in this amount not only adheres to the root surface but can also penetrate the root surface to a depth of about 10 μm, so it can remove most of the bacteria that have penetrated the root surface to a maximum depth of 8-9 μm.

Also, if doxycycline is added at less than 20 weight %, the bacteriostatic and root surface effects may be significantly reduced, and the content of the base excluding doxycycline increases relatively, increasing flowability. If the flowability of the periodontal disease treatment composition increases in this way, there may be a problem of not maintaining a minimum residence time of 5 minutes or more in the periodontal pocket.

In addition, if doxycycline is added in excess of 30 weight %, there is no significant difference in bacteriostatic and root surface effects, and it may affect the development of antibiotic resistance. And if doxycycline is added in excess of 30 weight %, it causes the formation of a large amount of crystals in the affected area due to supersaturation of doxycycline, and this crystal formation may cause problems that interfere with the healing of periodontal tissues.

Furthermore, if doxycycline is added in excess of 30 weight %, it may cause side effects such as killing not only harmful bacteria but also beneficial bacteria that help oral health, damaging tooth enamel, and discoloring teeth.

Meanwhile, physical methods can be used to remove contaminants from the root surface during periodontal disease treatment, but physical removal can make the surface of the root surface rough, which can serve as a reservoir for bacteria, so it is preferable to use chemical sterilization methods instead of physical removal.

The periodontal disease treatment composition according to the present invention includes an oral water-soluble carrier.

The oral water-soluble carrier is mixed with doxycycline to create a periodontal disease treatment composition, and includes a gelling agent and a moisturizer, and may optionally further include vegetable oil and water.

Such an oral water-soluble carrier adheres the periodontal disease treatment composition applied to the affected area and provides time for doxycycline to penetrate into the affected area.

Also, when the periodontal disease treatment composition is applied to the affected area in the oral cavity, it is preferable that the oral water-soluble carrier is configured to be removed from the oral cavity within 3 days, that is, about 72 hours at the latest, so as not to interfere with the formation and proliferation of granulation tissue.

This is because it is necessary for the water-soluble carrier to decompose in a short period, at the latest before about 3 days, after delivering doxycycline to the site where antibacterial action is needed, so as not to interfere with healing thereafter, to clean the root surface, and to naturally guide the patient's healing process.

Since macrophages, a type of immune system, appear from about 3 days after the treatment, and formation and proliferation of granulation tissue begin thereafter, it is preferable that the water-soluble carrier decomposes before that so as not to interfere with their action.

More preferably, it is appropriate for the water-soluble carrier to be maintained for about 5 to 10 minutes and decompose quickly. This is because if the water-soluble carrier decomposes before 5 minutes, it will not be able to sufficiently deliver and maintain doxycycline to the affected area, and after 10 minutes, although it has not entered the stage of macrophage appearance or full-scale healing process, there is no need for the water-soluble carrier to remain.

When using the water-soluble carrier according to the present invention, it is generally removed within about 10 minutes, at the latest within 2-3 hours after use, preventing side effects that interfere with the treatment or regeneration of tissues around the teeth that occur when the water-soluble carrier remains for a long time after delivering the antibiotic.

The oral water-soluble carrier can be added at 70 to 80 weight % based on 100 weight % of the periodontal disease treatment composition.

At this time, if the oral water-soluble carrier is added at less than 70 weight %, there may be a problem of reduced therapeutic effect due to ineffective delivery of doxycycline to the periodontal tissue, and the time that the periodontal disease treatment composition adheres to the periodontal pocket may be reduced, making it difficult to sterilize harmful bacteria that have penetrated the root surface.

And if the oral water-soluble carrier is added in excess of 80 weight %, there may be a problem of difficulty in obtaining an immediate therapeutic effect due to delayed release of doxycycline, and the risk of bacterial resistance may increase as the periodontal disease treatment composition stays in the periodontal pocket for too long.

In one embodiment, the oral water-soluble carrier according to the present invention may be composed of 5 to 35 weight % of a gelling agent and 65 to 95 weight % of a moisturizer based on a total of 100 weight % so that it can be decomposed within 72 hours in the oral cavity.

In another embodiment, the oral water-soluble carrier according to the present invention may be composed of 1 to 5 weight % of a gelling agent, 5 to 10 weight % of a moisturizer, 5 to 15 weight % of vegetable oil, and 70 to 85 weight % of water based on a total of 100 weight % so that it can be decomposed within 72 hours in the oral cavity. If necessary, the oral water-soluble carrier may further include 2 to 5 weight % of lecithin based on a total of 100 weight %.

The gelling agent acts to form a gel structure so that the periodontal disease treatment composition stays in the periodontal pocket for a certain time, increasing the physical stability of the emulsion to control the release of doxycycline.

For such a gelling agent, one or more of alginate, carboxymethylcellulose (CMC), guar gum, hydroxypropyl methylcellulose (HPMC) can be used, but it is preferable to use alginate so that it can be smoothly decomposed in the oral cavity.

The moisturizer inhibits the growth of oral bacteria by functioning similarly to saliva in the oral cavity, protects teeth and gums by maintaining moisture in the oral cavity, and performs the role of supplying and maintaining moisture to the oral mucosa to alleviate dryness.

For such a moisturizer, one or more of hyaluronic acid, propylene glycol, cetyl alcohol, polyethylene glycol, glycerin, sorbitol can be used, but it is preferable to use hyaluronic acid or propylene glycol so that it can be smoothly decomposed in the oral cavity.

The vegetable oil improves the solubility and permeability of the periodontal disease treatment composition, increases the solubility of the periodontal disease treatment composition to improve bioavailability, and performs the role of improving the user experience by softening the texture of the formulation. Also, when used with water, vegetable oil improves the overall stability of the formulation by increasing the stability of the oil-water interface.

For such vegetable oil, one or more of coconut oil, hinoki essential oil, oregano oil, olive oil, tea tree oil, eucalyptus oil, moringa oil, sesame oil, almond oil, grape seed oil can be used.

The water acts as a solvent, which is a basic medium for dissolving other components of the oral water-soluble carrier, and controls the viscosity of the periodontal disease treatment composition along with other components. At this time, distilled water, purified water, saline, etc. can be used as water.

The lecithin stably mixes vegetable oil and water to form an emulsion, promoting the penetration of doxycycline into the root surface.

Meanwhile, the oral water-soluble carrier according to the present invention may include 1 to 5 weight % of a pH-responsive polymer based on 100 weight % of the oral water-soluble carrier.

The pH-responsive polymer promotes the decomposition of the oral water-soluble carrier so that the oral water-soluble carrier that has delivered doxycycline to the root surface in the oral cavity can be completely decomposed within 24 hours, and it is stable at the pH in the tube (7-7.5) but swells in the slightly acidic environment (5.5-6.7) of the oral cavity to promote the decomposition of the oral water-soluble carrier.

For such a pH-responsive polymer, polyacrylic acid, carboxymethyl chitosan, polymethacrylic acid, alginate, etc. can be used.

As described above, although the oral water-soluble carrier is water-soluble, unlike doxycycline which is lipid-soluble, it is acceptable not to be formed with the same lipid solubility as doxycycline because it only needs to effectively deliver doxycycline to the periodontal tissue.

The periodontal disease treatment composition according to the present invention may further include a viscosity regulator.

The viscosity regulator increases the viscosity of the periodontal disease treatment composition so that the periodontal disease treatment composition can maintain an ointment-type formulation.

The viscosity regulator increases the viscosity so that the periodontal disease treatment composition lasts for at least 5 minutes on the surface of teeth and gums. Specifically, it is preferable that the viscosity of the periodontal disease treatment composition is in the range of 1500 to 2500 mPa·S.

For such a viscosity regulator, one or more selected from xanthan gum, guar gum, carrageenan, pectin, inulin, fructooligosaccharides (FOS), galactooligosaccharides (GOS), xylooligosaccharides (XOS), lactulose, resistant starch can be used.

Also, the viscosity regulator can be added at 3 to 20 parts by weight based on 100 parts by weight of the mixture of doxycycline and the oral water-soluble carrier. At this time, if the viscosity regulator is added at less than 3 parts by weight, there may be a problem of not maintaining a minimum residence time of 5 minutes or more in the periodontal pocket due to increased flowability. And if the viscosity regulator is added in excess of 20 parts by weight, there may be a problem of a large amount of waste due to clogging of the entrance when storing the periodontal disease treatment composition in a tube.

Meanwhile, it is preferable that the periodontal disease treatment composition according to the present invention is configured to be manufactured in an ointment form that secures convenience and simplicity of treatment and maintains rheological behavior that does not naturally flow down when stored in a tube but is only extruded by external force.

The periodontal disease treatment composition according to the present invention may further include additives.

The additives are added to the periodontal disease treatment composition within a range that does not damage the effects of the present invention, and one or more of antioxidants, water-soluble zinc salts, sweeteners, flavors, medicinal components, colorants, pH adjusters can be selectively used as appropriate considering the formulation. Of course, the additives are not limited to these.

Such additives can be added at 5 to 15 parts by weight based on 100 parts by weight of the mixture of doxycycline and the oral water-soluble carrier.

The antioxidant is added for the stability of the periodontitis treatment composition by inhibiting light-induced changes in doxycycline, and one or more selected from chitosan, tocopherol, sitosterol-containing extracts, unsaponifiable quantitative extracts of corn can be used.

The water-soluble zinc salt is a salt of zinc (Zn), which binds to enzymes used by anaerobic bacteria in the oral cavity, which are the cause of periodontal disease and bad breath, for energy metabolism, inhibiting enzyme activity, and inhibits the action of matrix metalloproteinases. As a result, the periodontal disease treatment composition containing water-soluble zinc salt can have an antibacterial action that inhibits the survival and growth of anaerobic bacteria, and can obtain a protective effect that inhibits the destruction of periodontal tissue.

For such water-soluble zinc salts, zinc oxide, zinc chloride (ZnC12), zinc hydroxide (Zn(OH)2), zinc sulfate (ZnSO4), or zinc gluconate can be used.

The sweetener gives a certain taste to the periodontal disease treatment composition and can also give a refreshing feeling. For such sweeteners, one or more selected from saccharin sodium, stevioside, neohesperidin dihydrochalcone, glycyrrhizin, perillartine, p-methoxycinnamic aldehyde, thaumatin, palatinose, maltitol, xylitol, arabitol can be used.

For flavors, one or more selected from natural flavors, single flavors, combination flavors, powder flavors can be used. Also, the flavor is not limited in its form and can be formed as essential oils, extracts, solids, and powders obtained by spray-drying any of these.

For medicinal components, one or more selected from tartar preventives such as condensed phosphates, ethane hydroxy diphosphonates, anti-inflammatory agents such as tranexamic acid, dipotassium glycyrrhizinate, ε-aminocaproic acid, phellodendron extract, coating agents such as hydroxyethylcellulose dimethyldiallyl ammonium chloride, astringents such as allantoin, allantoin chlorhydroxy aluminum, vitamin C, lysozyme chloride, glycyrrhetic acid and its salts, sodium chloride, and hypersensitivity inhibitors such as strontium chloride, potassium nitrate, aluminum lactate can be used. The content of such medicinal components can be varied within a range that does not damage the effects of the present invention.

For colorants, natural colorants such as safflower red pigment, gardenia yellow pigment, gardenia blue pigment, perilla pigment, red yeast pigment, red cabbage pigment, carrot pigment, hibiscus pigment, cocoa pigment, spirulina blue pigment, tamarind pigment, and legal colorants such as Red No. 3, Red No. 104, Red No. 105, Red No. 106, Yellow No. 4, Yellow No. 5, Green No. 3, Blue No. 1 can be used. The content of such colorants can be varied within a range that does not damage the effects of the present invention.

The pH adjuster adjusts the pH (20 °C) of the periodontal disease treatment composition to 7 to 7.5, and one or more selected from acetic acid, hydrochloric acid, sulfuric acid, nitric acid, citric acid, phosphoric acid, malic acid, gluconic acid, maleic acid, succinic acid, glutamic acid, sodium hydroxide, potassium hydroxide, sodium acetate, sodium carbonate, sodium citrate, sodium hydrogen citrate, sodium phosphate, disodium hydrogen phosphate can be used. The content of such pH adjusters can be varied within a range that does not damage the effects of the present invention.

Hereinafter, the present invention will be described more specifically through concrete examples and experimental examples. However, these examples and experimental examples are to help understand specific examples of the invention described above and should not be interpreted as limiting the scope of rights, etc.

### Example 1

(1) 50 g of alginate [Kimica, USA], 800 g of hyaluronic acid [Humedix, Korea], 150 g of coconut oil [LUONG QUOI COCONUT, Vietnam] were uniformly mixed for 30 minutes to obtain an oral water-soluble carrier.
(2) 250 g of doxycycline and 750 g of the aforementioned oral water-soluble carrier were uniformly mixed for 30 minutes to produce a periodontal disease treatment composition.

### Example 2

(1) A periodontal disease treatment composition was produced under the same conditions as Example 1, except that in the production of the oral water-soluble carrier, 100 g of alginate was used instead of 50 g of alginate, 650 g of hyaluronic acid was used instead of 800 g of hyaluronic acid, and 250 g of coconut oil was used instead of 150 g of coconut oil.
(2) Also, 260 g of doxycycline was used instead of 250 g of doxycycline, and 740 g of the oral water-soluble carrier from (1) was used instead of 750 g of the oral water-soluble carrier.

### Example 3

(1) 5 g of alginate [Kimica, USA], 50 g of hyaluronic acid [Humedix, Korea], 95 g of coconut oil [LUONG QUOI COCONUT, Vietnam], 850 g of purified water were uniformly mixed for 30 minutes to obtain an oral water-soluble carrier.
(2) 280 g of doxycycline and 720 g of the oral water-soluble carrier from (1) were uniformly mixed for 30 minutes to produce a periodontal disease treatment composition.

### Example 4

(1) 40 g of alginate [Kimica, USA], 60 g of hyaluronic acid [Humedix, Korea], 110 g of coconut oil [LUONG QUOI COCONUT, Vietnam], 780 g of purified water, 10 g of polyacrylic acid were uniformly mixed for 30 minutes to obtain an oral water-soluble carrier.
(2) 290 g of doxycycline and 710 g of the oral water-soluble carrier from (1) were uniformly mixed for 30 minutes to produce a periodontal disease treatment composition.

### Example 5

(1) A periodontal disease treatment composition was produced under the same conditions as Example 4, except that in the production of the oral water-soluble carrier, 30 g of polyacrylic acid was used instead of 10 g of polyacrylic acid, and 760 g of purified water was used instead of 780 g of purified water.
(2) 300 g of doxycycline and 700 g of the oral water-soluble carrier from (1) were uniformly mixed for 30 minutes to produce a periodontal disease treatment composition.

### Example 6

A periodontal disease treatment composition was produced under the same conditions as Example 1, except that 240 g of doxycycline was used instead of 250 g of doxycycline, and 760 g of the oral water-soluble carrier was used instead of 750 g of the oral water-soluble carrier.

### Example 7

(1) A periodontal disease treatment composition was produced under the same conditions as Example 3, except that in the production of the oral water-soluble carrier, 25 g of alginate was used instead of 5 g of alginate, 75 g of hyaluronic acid was used instead of 50 g of hyaluronic acid, and 50 g of coconut oil was used instead of 95 g of coconut oil.
(2) 200 g of doxycycline and 800 g of the oral water-soluble carrier from (1) were uniformly mixed for 30 minutes to produce a periodontal disease treatment composition.

### Comparative Example 1

A periodontal disease treatment composition was produced under the same conditions as Example 1, except that 190 g of doxycycline was used instead of 250 g of doxycycline, and 810 g of the oral water-soluble carrier was used instead of 750 g of the oral water-soluble carrier.

### Comparative Example 2

(1) A periodontal disease treatment composition was produced under the same conditions as Example 3, except that in the production of the oral water-soluble carrier, 50 g of alginate was used instead of 5 g of alginate, 100 g of hyaluronic acid was used instead of 50 g of hyaluronic acid, 150 g of coconut oil was used instead of 95 g of coconut oil, and 700 g of purified water was used instead of 850 g of purified water.
(2) 180 g of doxycycline and 820 g of the oral water-soluble carrier from (1) were uniformly mixed for 30 minutes to produce a periodontal disease treatment composition.

### Comparative Example 3

(1) 30 g of alginate [Kimica, USA], 70 g of hyaluronic acid [Humedix, Korea], 90 g of coconut oil [LUONG QUOI COCONUT, Vietnam], 775 g of purified water, 35 g of lecithin were uniformly mixed for 30 minutes to obtain an oral water-soluble carrier.
(2) 170 g of doxycycline and 830 g of the oral water-soluble carrier from (1) were uniformly mixed for 30 minutes to produce a periodontal disease treatment composition.

### Comparative Example 4

(1) A periodontal disease treatment composition was produced under the same conditions as Example 4, except that in the production of the oral water-soluble carrier, 50 g of polyacrylic acid was used instead of 10 g of polyacrylic acid, and 740 g of purified water was used instead of 780 g of purified water.
(2) 310 g of doxycycline and 690 g of the oral water-soluble carrier from (1) were uniformly mixed for 30 minutes to produce a periodontal disease treatment composition.

### Comparative Example 5

A water-soluble carrier was prepared under the same conditions as Comparative Example 4, and 320 g of doxycycline and 680 g of the obtained oral water-soluble carrier were uniformly mixed for 30 minutes to produce a pharmaceutical composition for periodontal disease treatment.

### Comparative Example 6

300 g of doxycycline and 700 g of polycaprolactone [Polycaprolactone, Sigma-Aldrich] were uniformly mixed for 30 minutes to produce a periodontal disease treatment composition.

### Comparative Example 7

200 g of doxycycline and 800 g of polycaprolactone [Polycaprolactone, Sigma-Aldrich] were uniformly mixed for 30 minutes to produce a periodontal disease treatment composition.

Hereinafter, the amounts of doxycycline and carrier used in Examples 1 to 7 and Comparative Examples 1 to 7 are summarized in [Table 1], and the composition of the water-soluble carrier is summarized in [Table 2].

TABLE 1
Amounts of Doxycycline and Carrier Used (unit: g)

| Example/ | | Water-soluble | Conventional |
|---|---|---|---|
| Comp. Ex. | Doxycycline(g) | carrier (g) | carrier(g) |
| Example 1 | 250 | 750 | |
| Example 2 | 260 | 740 | |
| Example 3 | 280 | 720 | |
| Example 4 | 290 | 710 | |
| Example 5 | 300 | 700 | |
| Example 6 | 240 | 760 | |
| Example 7 | 200 | 800 | |
| Comp. Ex. 1 | 190 | 810 | |
| Comp. Ex. 2 | 180 | 820 | |
| Comp. Ex. 3 | 170 | 830 | |
| Comp. Ex. 4 | 310 | 690 | |
| Comp. Ex. 5 | 320 | 680 | |
| Comp. Ex. 6 | 300 | | 700 |
| Comp. Ex. 7 | 200 | | 800 |

TABLE 2
Composition and Amount of Water-Soluble Carrier (unit: g)

| | | Hyaluronic | Coconut | Purified | | Polyacrylic |
|---|---|---|---|---|---|---|
| | Alginate | acid | oil | water | Lecithin | acid |
| Example 1 | 50 | 800 | 150 | | | |
| Example 2 | 100 | 650 | 250 | | | |
| Example 3 | 5 | 50 | 95 | 850 | | |
| Example 4 | 40 | 60 | 110 | 780 | | 10 |
| Example 5 | 40 | 60 | 110 | 760 | | 30 |
| Example 6 | 50 | 800 | 150 | | | |
| Example 7 | 25 | 75 | 50 | 850 | | |
| Comp. Ex. 1 | 50 | 800 | 150 | | | |
| Comp. Ex. 2 | 50 | 100 | 150 | 700 | | |
| Comp. Ex. 3 | 30 | 70 | 90 | 775 | 35 | |
| Comp. Ex. 4 | 40 | 60 | 110 | 740 | | 50 |
| Comp. Ex. 5 | 40 | 60 | 110 | 740 | | 50 |

### Experimental Example

Among patients with periodontitis who had been treated with the periodontal disease treatment compositions produced according to Examples 1 to 7 and Comparative Examples 1 to 7, patients who had passed more than 1 year after treatment were selected through interviews and records to identify patients who had not had additional treatments in the same area after past periodontitis treatment.

To check the degree of inflammation when the patient first visited for past periodontitis treatment, the depth (Probing Depth) that a probe entered between the tooth and the inflamed gum was compared with the depth that a probe entered the same area at a point at least 1 year later, and the depth (mm) was summarized in [Table 3] below. At this time, a “UNC-15 periodontal probe (Hu-Friedy, Chicago, IL, USA)” was used to measure the clinical indicator PD (Probing Depth).

In [Table 3] below, the recovery rate was calculated as (pre-treatment depth-treatment depth)/pre-treatment depth.

TABLE 3
Recovery Rate After More Than 1 Year of Long-Term Follow-Up

| | | | Date of | Date of | PD | PD | |
|---|---|---|---|---|---|---|---|
| Example/ | Gender | | examination | examination | Before | After | |
| Comp. | (Male/ | | before | after | Treatment | Treatment | Recovery |
| Ex. | Female) | Age | treatment | treatment | (mm) | (mm) | Rate |
| Example 1 | F | 68 | 18 Jan. 2022 | 29 Mar. 2024 | 7 | 0.5 | 0.93 |
| Example | F | 58 | 5 Dec. 2019 | 4 Aug. 2022 | 8 | 0.5 | 0.94 |
| 2-A | | | | | | | |
| Example | F | 58 | 5 Dec. 2019 | 4 Aug. 2022 | 7.5 | 0.5 | 0.93 |
| 2-B | | | | | | | |
| Example | F | 58 | 5 Dec. 2019 | 4 Aug. 2022 | 7 | 0 | 1.00 |
| 2-C | | | | | | | |
| Example 3 | M | 53 | 29 Jun. 2020 | 3 May 2023 | 6 | 0 | 1.00 |
| Example 4 | M | 48 | 16 Jun. 2022 | 1 Dec. 2023 | 8 | 0 | 1.00 |
| Example 5 | F | 37 | 28 Dec. 2021 | 30 May 2024 | 6 | 0 | 1.00 |
| Example 6 | M | 61 | 14 Jan. 2022 | 14 Sep. 2023 | 8.8 | 1.5 | 0.83 |
| Example | F | 53 | 13 Jun. 2020 | 11 Feb. 2023 | 6 | 1 | 0.83 |
| 7-A | | | | | | | |
| Example | F | 53 | 13 Jun. 2020 | 11 Feb. 2023 | 6 | 1 | 0.83 |
| 7-B | | | | | | | |
| Comp. Ex. | M | 63 | 10 Mar. 2018 | 15 Apr. 2024 | 8 | 3 | 0.63 |
| 1-A | | | | | | | |
| Comp. Ex. | M | 63 | 10 Mar. 2018 | 15 Apr. 2024 | 8 | 4 | 0.50 |
| 1-B | | | | | | | |
| Comp. Ex. | F | 41 | 11 Aug. 2018 | 16 Feb. 2023 | 7 | 3 | 0.57 |
| 2 | | | | | | | |
| Comp. Ex. | F | 60 | 19 Jul. 2019 | 20 Apr. 2021 | 8 | 2.5 | 0.69 |
| 3 | | | | | | | |
| Comp. Ex. | M | 50 | 16 Jun. 2022 | 1 Dec. 2024 | 7 | 0 | 1.00 |
| 4 | | | | | | | |
| Comp. Ex. | F | 50 | 13 Jan. 2020 | 9 May 2024 | 7.5 | 0.5 | 0.93 |
| 5 | | | | | | | |
| Comp. Ex. | M | 52 | 20 Apr. 2018 | 11 Jun. 2024 | 7.5 | 2 | 0.73 |
| 6 | | | | | | | |
| Comp. Ex. | M | 49 | 14 Feb. 2019 | 9 Nov. 2023 | 7 | 1.5 | 0.79 |
| 7-A | | | | | | | |
| Comp. Ex. | M | 49 | 14 Feb. 2019 | 9 Nov. 2023 | 7.5 | 2 | 0.73 |
| 7-B | | | | | | | |

Also, panoramic photos were taken (using Vatech's PaX-i3D SMART) at the time when the patient first visited with periodontal disease and after the periodontal disease was treated and the gum tissue regeneration was completed (generally taking about 1 year).

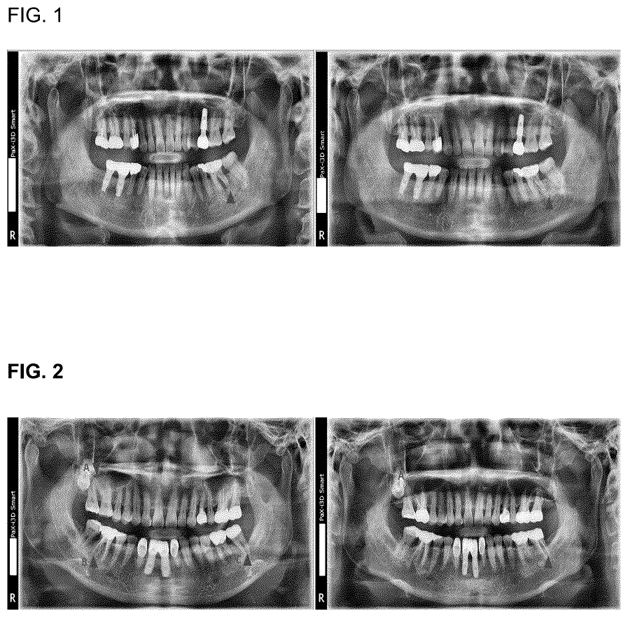

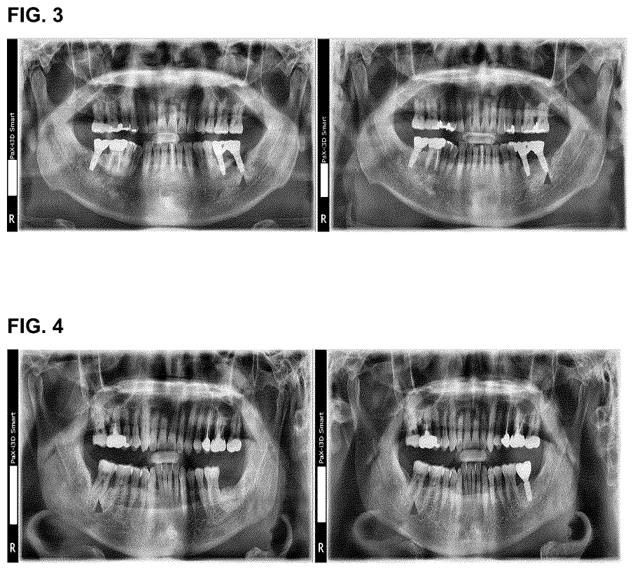

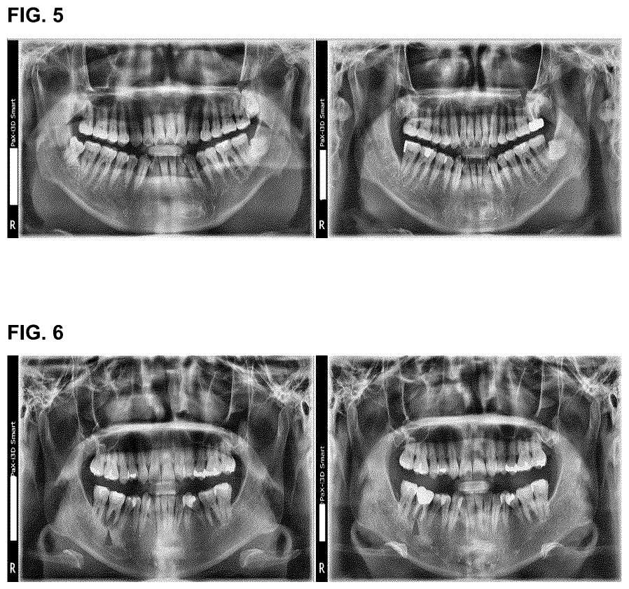

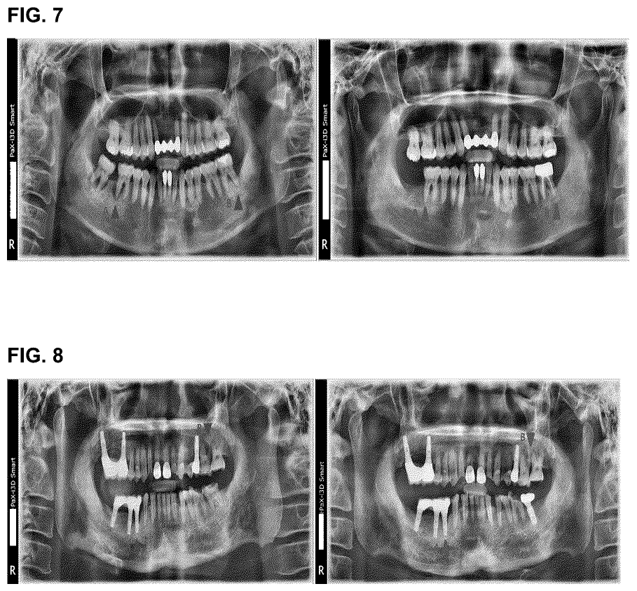

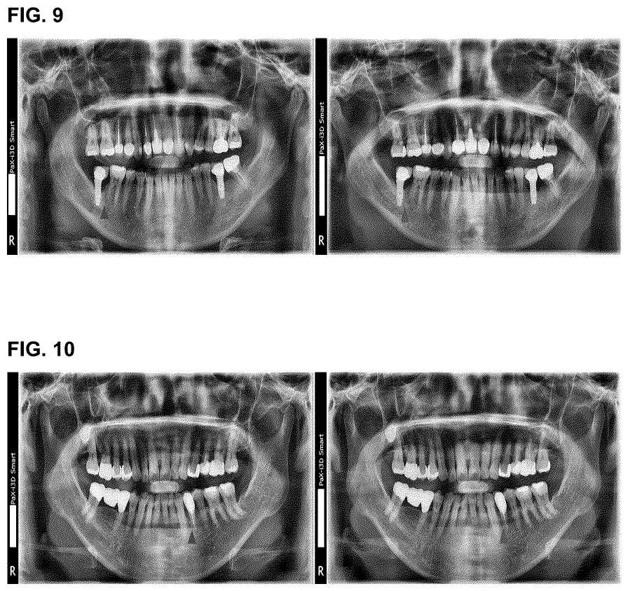

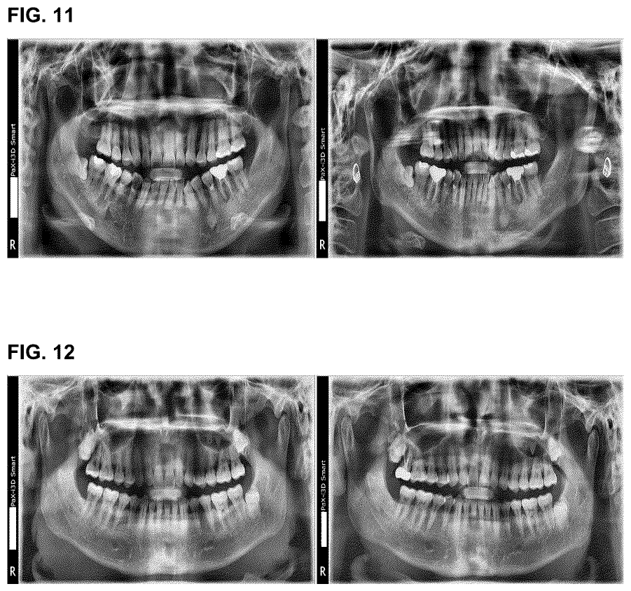

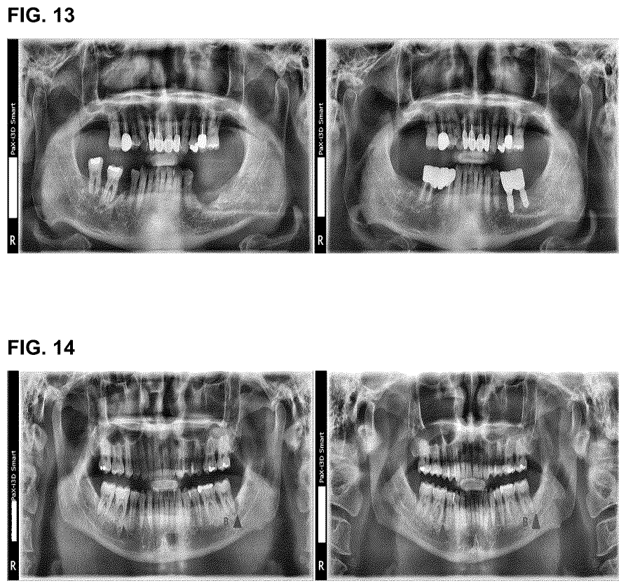

Comparative photos before and after treatment at a point more than 1 year after treatment according to Examples 1 to 7 are shown in FIGS. 1 to 7. Also, comparative photos according to Comparative Examples 1 to 7 are shown in FIGS. 8 to 14 in the same way. At this time, the affected area in the panoramic photo was marked with a red triangle.

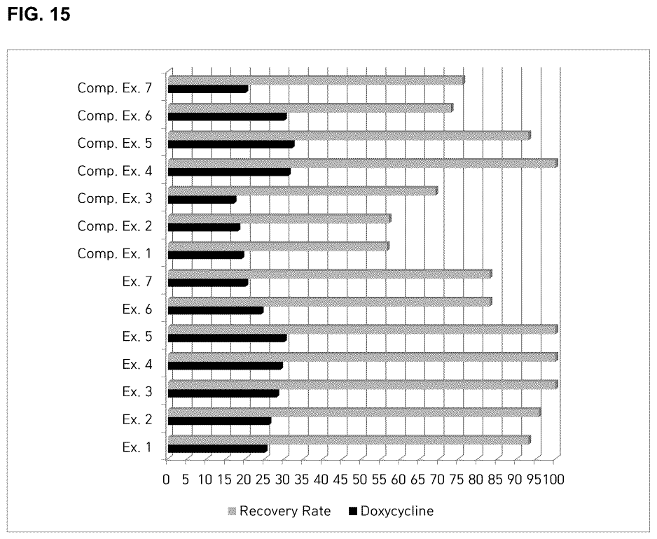

FIG. 15 is a graph showing the average recovery rate by concentration of doxycycline included in the pharmaceutical composition for treating periodontal disease according to the present invention.

Referring to [Table 3] and FIG. 15, it was found that when the concentration of doxycycline was 20 to 30 weight %, especially 25 to 30 weight %, granulation tissue was formed and proliferated after a long period, and the gum tissue recovered to normal.

Also, looking at cases where the concentration of doxycycline is outside the range of 20 to 30 weight % (Comparative Examples 1-3) and cases where a water-soluble carrier is not used (Comparative Examples 6-7), it was found that the average recovery rate was less than 0.70, indicating that the gum tissue did not recover sufficiently.

Furthermore, in cases where the concentration of doxycycline exceeded 30 weight % (Comparative Examples 4-5), although the gum tissue recovered to normal, there was a problem of increased difficulty in managing the periodontal disease treatment composition due to the formation of a large amount of crystals caused by supersaturation of doxycycline.

As a result, it was observed that superior periodontal disease treatment effects were provided when using a periodontal disease treatment composition containing doxycycline with a concentration of 20 to 30 weight % and a water-soluble carrier.

While the present invention has been described with reference to preferred embodiments, those skilled in the art will understand that the present invention can be variously modified and changed within the scope of the present invention as defined in the following claims without departing from the spirit and scope of the present invention.

1. A water-soluble carrier-based high-concentration doxycycline-containing pharmaceutical composition for periodontal disease treatment, comprising:
20 to 30 weight % of doxycycline; and
70 to 80 weight % of an oral water-soluble carrier,
wherein the oral water-soluble carrier comprises 1 to 5 weight % of alginate, 5 to 10 weight % of hyaluronic acid, 5 to 15 weight % of coconut oil, and 70 to 85 weight % of purified water.

2. The water-soluble carrier-based high-concentration doxycycline-containing pharmaceutical composition for periodontal disease treatment according to claim 1, comprising:
25 to 30 weight % of doxycycline; and
70 to 75 weight % of an oral water-soluble carrier.

3. The water-soluble carrier-based high-concentration doxycycline-containing pharmaceutical composition for periodontal disease treatment according to claim 1, wherein the oral water-soluble carrier is removed within 72 hours after application.

4. The water-soluble carrier-based high-concentration doxycycline-containing pharmaceutical composition for periodontal disease treatment according to claim 1, wherein the oral water-soluble carrier is removed between 5 minutes and 10 minutes after application.

5-8. (canceled)

9. The water-soluble carrier-based high-concentration doxycycline-containing pharmaceutical composition for periodontal disease treatment according to claim 1, wherein the oral water-soluble carrier further comprises 1 to 5 weight % of polyacrylic acid as a pH-responsive polymer.
